(19) United States
(12) Patent Application Publication
(10) Pub. No.: US 2016/0045513 A1
(43) Pub. Date: Feb. 18, 2016

(54) ISOXAZOLE COMPOUND FOR THE TREATMENT OF CANCER

(71) Applicants: Patrick Chene, Mulhouse (FR); Carlos Garcia-Echeverria, Basel (CH); Michael Rugaard Jensen, Basel (CH); Cornelia Quadt, Allschwil (CH); Thomas Radimerski, Tecknau (CH); Joseph Schoepfer, Riehen (CH)

(72) Inventors: Patrick Chene, Mulhouse (FR); Carlos Garcia-Echeverria, Basel (CH); Michael Rugaard Jensen, Basel (CH); Cornelia Quadt, Allschwil (CH); Thomas Radimerski, Tecknau (CH); Joseph Schoepfer, Riehen (CH)

(73) Assignee: Novartis AG, Basel (CH)

(21) Appl. No.: 14/925,263

(22) Filed: Oct. 28, 2015

Related U.S. Application Data
(63) Continuation of application No. 14/267,946, filed on May 2, 2014.
Application No. 14/267,946 is a continuation of application No. 13/461,855, filed on May 2, 2012, now abandoned.
Application No. 13/461,855 is a continuation of application No. 13/314,584, filed on Dec. 8, 2011, now abandoned.
Application No. 13/314,584 is a continuation of application No. 12/680,657, filed on Mar. 29, 2010, now abandoned.

(30) Foreign Application Priority Data
Oct. 12, 2007 (EP) 07118421.2

(51) Int. Cl. A61K 31/5377

(52) U.S. Cl. CPC A61K 31/5377

(57) ABSTRACT

The use of 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate for the treatment of cancer of the bladder, the colon, the liver, the lung, the breast, the vagina, the ovaries, the pancreas, the kidney, the stomach, the gastrointestinal tract, the prostate, the head and neck, the peritoneal cavity, the thyroid, the bone, the brain, the central nervous system and/or the blood and/or for the treatment of myelodysplastic syndrome, systemic mastocytosis, von Hippel-Lindau syndrome, multicentric Castleman disease and/or psioriasis.

This is a continuation of application Ser. No. 14/267946 filed on May 2, 2014, which is a continuation of application Ser. No. 13/461855 filed on May 2, 2012, which is a continuation of Ser. No. 13/314584 filed on Dec. 8, 2011, which is a continuation of Ser. No. 12/680,657 filed on Mar. 29, 2010, which is a National Stage of International Application No. PCT/EP2008/063605 filed on Oct. 10, 2008, which claims priority under 35 U.S.C. §119 to EP Application Serial No. 07118421.2 filed Oct. 12, 2007, which in its entirety are herein incorporated by reference.

The invention relates to the use of 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate for the manufacture of pharmaceutical compositions for use in the treatment of cancer, e.g. solid tumors, e.g. sarcomas, e.g. carcinomas of the bladder, the colon, the liver, the lung, e.g. pleural mesothelioma, e.g. non small cell, e.g. small cell, the breast, the vagina, the ovaries, the pancreas, the kidney, the stomach, gastrointestinal tract, e.g. gastrointestinal stromal tumor, e.g. the small intestine, e.g. the esophagus, e.g. the bile duct, the prostate, the head and neck, the peritoneal cavity, the thyroid, the bone, the brain, the central nervous system e.g. glioblastoma, e.g. neuroblastoma, and/or melanoma and/or cancer of the blood , e.g. hematological cancer, e.g. leukemia, e.g. acute myeloid leukemia, e.g. chronic myeloid leukemia, e.g. chronic lymphatic leukemia, e.g. acute lymphatic leukemia, e.g. multiple myeloma e.g. lymphomas, and/or for use in treatment of myelodysplastic syndrome, systemic mastocytosis, von Hippel-Lindau syndrome, multicentric Castleman disease and/or psioriasis, to the use of 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate in the treatment of cancer, e.g. solid tumors, e.g. sarcomas, e.g. carcinomas of the bladder, the colon, the liver, the lung, e.g. pleural mesothelioma, e.g. non small cell, e.g. small cell, the breast, the vagina, the ovaries, the pancreas, the kidney, the stomach, gastrointestinal tract, e.g. gastrointestinal stromal tumor, e.g. the small intestine, e.g. the esophagus, e.g. the bile duct, the prostate, the head and neck, the peritoneal cavity, the thyroid, the bone, the brain, the central nervous system e.g. glioblastoma, e.g. neuroblastoma, and/or melanoma and/or cancer of the blood , e.g. hematological cancer, e.g. leukemia, e.g. acute myeloid leukemia, e.g. chronic myeloid leukemia, e.g. chronic lymphatic leukemia, e.g. acute lymphatic leukemia, e.g. multiple myeloma e.g. lymphomas, and/or in the treatment of myelodysplastic syndrome, systemic mastocytosis, von Hippel-Lindau syndrome, multicentric Castleman disease and/or psioriasis, and to a method of treating warm-blooded animals including humans suffering from cancer, e.g. solid tumors, e.g. sarcomas, e.g. carcinomas of the bladder, the colon, the liver, the lung, e.g. pleural mesothelioma, e.g. non small cell, e.g. small cell, the breast, the vagina, the ovaries, the pancreas, the kidney, the stomach, gastrointestinal tract, e.g. gastrointestinal stromal tumor, e.g. the small intestine, e.g. the esophagus, e.g. the bile duct, the prostate, the head and neck, the peritoneal cavity, the thyroid, the bone, the brain, the central nervous system e.g. glioblastoma, e.g. neuroblastoma, and/or melanoma and/or cancer of the blood , e.g. hematological cancer, e.g. leukemia, e.g. acute myeloid leukemia, e.g. chronic myeloid leukemia, e.g. chronic lymphatic leukemia, e.g. acute lymphatic leukemia, e.g. multiple myeloma e.g. lymphomas, and/or myelodysplastic syndrome, systemic mastocytosis, von Hippel-Lindau syndrome, multicentric Castleman disease and/or psioriasis by administering to said animal in need of such treatment an effective dose of 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate.

Management of cancer, e.g. solid tumors, e.g. sarcomas, e.g. carcinomas of the bladder, the colon, the liver, the lung, e.g. pleural mesothelioma, e.g. non small cell, e.g. small cell, the breast, the vagina, the ovaries, the pancreas, the kidney, the stomach, gastrointestinal tract, e.g. gastrointestinal stromal tumor, e.g. the small intestine, e.g. the esophagus, e.g. the bile duct, the prostate, the head and neck, the peritoneal cavity, the thyroid, the bone, the brain, the central nervous system e.g. glioblastoma, e.g. neuroblastoma, and/or melanoma and/or cancer of the blood , e.g. hematological cancer, e.g. leukemia, e.g. acute myeloid leukemia, e.g. chronic myeloid leukemia, e.g. chronic lymphatic leukemia, e.g. acute lymphatic leukemia, e.g. multiple myeloma e.g. lymphomas, and/or myelodysplastic syndrome, systemic mastocytosis, von Hippel-Lindau syndrome, multicentric Castleman disease and/or psioriasis is a major problem.

Heat shock protein 90 (Hsp90) is recognized as a new anti-cancer target. Hsp90 is a ubiquitous, highly abundant (1-2% of the total cellular protein), essential protein which functions as a molecular chaperone to ensure the conformational stability, shape and function of client proteins. Inhibition of its intrinsic ATPase activity of Hsp90 disrupts the Hsp90-client protein interaction resulting in their degradation via the ubiquitin proteasome pathway. A subset of Hsp90 client proteins, such as Raf, AKT, CDK4 and the EGFR family including ErbB2 are oncogenic signaling molecules critically involved in cell growth, differentiation and apoptosis, processes which are fundamentaly important in cancer cells. The simultaneous degradation of multiple oncoproteins is believed to produce the anti-tumor effects observed with Hsp90 inhibitors.

The Hsp90 family of chaperones is comprised of four members: Hsp90a and Hsp90β both located in the cytosol, GRP94 in the endoplasmic reticulum, and TRAP1 in the mitochondria (Csermely et al., 1998). Hsp90 is the most abundant cellular chaperone, constituting about 1% -2% of total protein (Jakob and Buchner, 1994). Among the stress proteins, Hsp90 is unique because it is not required for the biogenesis of most polypeptides (Nathan et al., 1997). Its cellular targets, also called client proteins, are conformationally labile signal transducers that play a critical role in growth control, cell survival and tissue development (Pratt and Toft, 2003).

Hsp90 chaperones, which possess a conserved ATP-binding site at their N-terminal domain (Chene, 2002) belong to a small ATPase sub-family known as the DNA Gyrase, Hsp90, Histidine Kinase and MutL (GHKL) sub-family (Dutta and Inouye, 2000). The chaperoning (folding) activity of Hsp90 depends on its ATPase activity which is weak for the isolated enzyme. However, it has been shown that the ATPase activity of Hsp90 is enhanced upon its association with proteins known as co-chaperones (Kamal et al., 2003). Therefore, in vivo, Hsp90 proteins work as subunits of large, dynamic protein complexes. Hsp90 is essential for eukaryotic cell survival and is overexpressed in many tumors.

5-(2,4-Dihydroxy-5-isopropyl-phenyl)-4-(4-morpholin-4-ylmethyl-phenyl)-isoxazole-3-carboxylic acid ethylamide is an Hsp90 inhibitor, its synthesis is described for instance in WO 2004/072051, example 78, included herein by reference.

Surprisingly it has now been found that 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate is useful in the treatment of cancer, e.g. solid tumors, e.g. sarcomas, e.g. carcinomas of the bladder, the colon, the liver, the lung, e.g. pleural mesothelioma, e.g. non small cell, e.g. small cell, the breast, the vagina, the ovaries, the pancreas, the kidney, the stomach, gastrointestinal tract, e.g. gastrointestinal stromal tumor, e.g. the small intestine, e.g. the esophagus, e.g. the bile duct, the prostate, the head and neck, the peritoneal cavity, the thyroid, the bone, the brain, the central nervous system e.g. glioblastoma, e.g. neuroblastoma, and/or melanoma and/or cancer of the blood , e.g. hematological cancer, e.g. leukemia, e.g. acute myeloid leukemia, e.g. chronic myeloid leukemia, e.g. chronic lymphatic leukemia, e.g. acute lymphatic leukemia, e.g. multiple myeloma e.g. lymphomas, and/or in treating of myelodysplastic syndrome, systemic mastocytosis, von Hippel-Lindau syndrome, multicentric Castleman disease and/or psioriasis.

Accordingly the present invention provides the use of 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate for the manufacture of pharmaceutical compositions for use in the treatment of cancer, e.g. solid tumors, e.g. sarcomas, e.g. carcinomas of the bladder, the colon, the liver, the lung, e.g. pleural mesothelioma, e.g. non small cell, e.g. small cell, the breast, the vagina, the ovaries, the pancreas, the kidney, the stomach, gastrointestinal tract, e.g. gastrointestinal stromal tumor, e.g. the small intestine, e.g. the esophagus, e.g. the bile duct, the prostate, the head and neck, the peritoneal cavity, the thyroid, the bone, the brain, the central nervous system e.g. glioblastoma, e.g. neuroblastoma, and/or melanoma and/or cancer of the blood , e.g. hematological cancer, e.g. leukemia, e.g. acute myeloid leukemia, e.g. chronic myeloid leukemia, e.g. chronic lymphatic leukemia, e.g. acute lymphatic leukemia, e.g. multiple myeloma e.g. lymphomas, and/or for use in treatment of myelodysplastic syndrome, systemic mastocytosis, von Hippel-Lindau syndrome, multicentric Castleman disease and/or psioriasis.

In another aspect the present invention provides the use of 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate in the treatment of cancer, e.g. solid tumors, e.g. sarcomas, e.g. carcinomas of the bladder, the colon, the liver, the lung, e.g. pleural mesothelioma, e.g. non small cell, e.g. small cell, the breast, the vagina, the ovaries, the pancreas, the kidney, the stomach, gastrointestinal tract, e.g. gastrointestinal stromal tumor, e.g. the small intestine, e.g. the esophagus, e.g. the bile duct, the prostate, the head and neck, the peritoneal cavity, the thyroid, the bone, the brain, the central nervous system e.g. glioblastoma, e.g. neuroblastoma, and/or melanoma and/or cancer of the blood , e.g. hematological cancer, e.g. leukemia, e.g. acute myeloid leukemia, e.g. chronic myeloid leukemia, e.g. chronic lymphatic leukemia, e.g. acute lymphatic leukemia, e.g. multiple myeloma e.g. lymphomas, and/or in the treatment of myelodysplastic syndrome, systemic mastocytosis, von Hippel-Lindau syndrome, multicentric Castleman disease and/or psioriasis.

In a further aspect the present invention provides 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate for use in treating cancer, e.g. solid tumors, e.g. sarcomas, e.g. carcinomas of the bladder, the colon, the liver, the lung, e.g. pleural mesothelioma, e.g. non small cell, e.g. small cell, the breast, the vagina, the ovaries, the pancreas, the kidney, the stomach, gastrointestinal tract, e.g. gastrointestinal stromal tumor, e.g. the small intestine, e.g. the esophagus, e.g. the bile duct, the prostate, the head and neck, the peritoneal cavity, the thyroid, the bone, the brain, the central nervous system e.g. glioblastoma, e.g. neuroblastoma, and/or melanoma and/or cancer of the blood, e.g. hematological cancer, e.g. leukemia, e.g. acute myeloid leukemia, e.g. chronic myeloid leukemia, e.g. chronic lymphatic leukemia, e.g. acute lymphatic leukemia, e.g. multiple myeloma e.g. lymphomas, and/or for use in treating myelodysplastic syndrome, systemic mastocytosis, von Hippel-Lindau syndrome, multicentric Castleman disease and/or psioriasis.

In a further aspect the present invention provides a method of treating humans suffering from cancer, e.g. solid tumors, e.g. sarcomas, e.g. carcinomas of the bladder, the colon, the liver, the lung, e.g. pleural mesothelioma, e.g. non small cell, e.g. small cell, the breast, the vagina, the ovaries, the pancreas, the kidney, the stomach, gastrointestinal tract, e.g. gastrointestinal stromal tumor, e.g. the small intestine, e.g. the esophagus, e.g. the bile duct, the prostate, the head and neck, the peritoneal cavity, the thyroid, the bone, the brain, the central nervous system e.g. glioblastoma, e.g. neuroblastoma, and/or melanoma and/or cancer of the blood , e.g. hematological cancer, e.g. leukemia, e.g. acute myeloid leukemia, e.g. chronic myeloid leukemia, e.g. chronic lymphatic leukemia, e.g. acute lymphatic leukemia, e.g. multiple myeloma e.g. lymphomas, and/or myelodysplastic syndrome, systemic mastocytosis, von Hippel-Lindau syndrome, multicentric Castleman disease and/or psioriasis which comprises administering to said human in need of such treatment a dose of 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate effective against cancer, e.g. solid tumors, e.g. sarcomas, e.g. carcinomas of the bladder, the colon, the liver, the lung, e.g. pleural mesothelioma, e.g. non small cell, e.g. small cell, the breast, the vagina, the ovaries, the pancreas, the kidney, the stomach, gastrointestinal tract, e.g. gastrointestinal stromal tumor, e.g. the small intestine, e.g. the esophagus, e.g. the bile duct, the prostate, the head and neck, the peritoneal cavity, the thyroid, the bone, the brain, the central nervous system e.g. glioblastoma, e.g. neuroblastoma, and/or melanoma and/or cancer of the blood , e.g. hematological cancer, e.g. leukemia, e.g. acute myeloid leukemia, e.g. chronic myeloid leukemia, e.g. chronic lymphatic leukemia, e.g. acute lymphatic leukemia, e.g. multiple myeloma e.g. lymphomas, and/or myelodysplastic syndrome, systemic mastocytosis, von Hippel-Lindau syndrome, multicentric Castleman disease and/or psioriasis.

In a further aspect the present invention provides a pharmaceutical preparation for the treatment of cancer, e.g. solid tumors, e.g. sarcomas, e.g. carcinomas of the bladder, the colon, the liver, the lung, e.g. pleural mesothelioma, e.g. non small cell, e.g. small cell, the breast, the vagina, the ovaries, the pancreas, the kidney, the stomach, gastrointestinal tract, e.g. gastrointestinal stromal tumor, e.g. the small intestine, e.g. the esophagus, e.g. the bile duct, the prostate, the head and neck, the peritoneal cavity, the thyroid, the bone, the brain, the central nervous system e.g. glioblastoma, e.g. neuroblastoma, and/or melanoma and/or cancer of the blood , e.g. hematological cancer, e.g. leukemia, e.g. acute myeloid leukemia, e.g. chronic myeloid leukemia, e.g. chronic lymphatic leukemia, e.g. acute lymphatic leukemia, e.g. multiple myeloma e.g. lymphomas, and/or myelodysplastic syndrome, systemic mastocytosis, von Hippel-Lindau syndrome, multicentric Castleman disease and/or psioriasis comprising 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate and at least one pharmaceutically acceptable carrier.

Depending on species, age, individual condition, mode of administration, and the clinical picture in question, effective doses for example weekly doses of about 2 to 300 mg, preferably 50 to 160 mg of 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate are administered to a human.

The present invention further provides a method for administering to a human having cancer, e.g. solid tumors, e.g. sarcomas, e.g. carcinomas of the bladder, the colon, the liver, the lung, e.g. pleural mesothelioma, e.g. non small cell, e.g. small cell, the breast, the vagina, the ovaries, the pancreas, the kidney, the stomach, gastrointestinal tract, e.g. gastrointestinal stromal tumor, e.g. the small intestine, e.g. the esophagus, e.g. the bile duct, the prostate, the head and neck, the peritoneal cavity, the thyroid, the bone, the brain, the central nervous system e.g. glioblastoma, e.g. neuroblastoma, and/or melanoma and/or cancer of the blood , e.g. hematological cancer, e.g. leukemia, e.g. acute myeloid leukemia, e.g. chronic myeloid leukemia, e.g. chronic lymphatic leukemia, e.g. acute lymphatic leukemia, e.g. multiple myeloma e.g. lymphomas, and/or myelodysplastic syndrome, systemic mastocytosis, von Hippel-Lindau syndrome, multicentric Castleman disease and/or psioriasis 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate, which comprises administering a pharmaceutically effective amount of 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate to a human subject about once weekly or more frequently.

Following is a description by way of example only.

### EXAMPLE 1

### In Vitro Effects of 5-(2,4-Dihydroxy-5-isopropyl-phenyl)-4-(4-morpholin-4-ylmethyl-phenyl)-isoxazole-3-Carboxylic Acid Ethylamide (AUY922) on a Panel of Tumor Derived Cell Lines

Thirty-seven cancer derived cell lines are used (BT474, MDA-MB-361, MDA-MB-453, SKBr3, T47D, MCF7, MDA-MB-231, MDA-MB-468, SK-MEL-5, A375, MALME-3M, SK-MEL-28, WM266.4, RPMI8226, U266, BE, Colo205, HCT116, HT29, MAWI, RKO, U87MG, HN5, RPMI-8226, A549, MV522, NCI-H1299, NCI-H460, 41M, A2780, CHI, NCI-N87, SKOV3, PC3, MO7e, GIST882 and Baf3) to test the effect of 5-(2,4-Dihydroxy-5-isopropyl-phenyl)-4-(4-morpholin-4-ylmethyl-phenyl)-isoxazole-3-carboxylic acid ethylamide. Cell lines are commercially available from American Type Culture Collection (ATCC). These cell lines cover the following 12 cancer or tumor types: breast, melanoma, multiple myeloma (MM), colon, glioblastoma, head & neck, leukemia, lung, ovarian, prostate, stomach and gastrointestinal stromal tumour (GIST). After division and medium change, cells from stock culture are seeded on cell plates and cultured for about 18 hours to allow cell growth and attachment before starting the assay. On the first day of the assay, 5-(2,4-Dihydroxy-5-isopropyl-phenyl)-4-(4-morpholin-4-ylmethyl-phenyl)-isoxazole-3-carboxylic acid ethylamide is added to the medium at various concentrations up to 10 p. Cells are cultured up to 72 or 96 hours and cell proliferation is determined using commercially available cell proliferation kits.

Table 1 shows the concentrations (nM) of 5-(2,4-Dihydroxy-5-isopropyl-phenyl)-4-(4-morpholin-4-ylmethyl-phenyl)-isoxazole-3-carboxylic acid ethylamide which inhibit cell proliferation by 50% (IC₅₀). The cells were continually exposed to 5-(2,4-Dihydroxy-5-isopropyl-phenyl)-4-(4-morpholin-4-ylmethyl-phenyl)-isoxazole-3-carboxylic acid ethylamide for either 72 or 96 hours and cell growth was determined by commercially available kits based on either SRB, Alamar blue, methylene blue or WST-1 methods.

TABLE 1

| Tumor type | Cell line | IC₅₀ (nM) |
|---|---|---|
| Breast | BT474 | 2.8 |
| | MDA-MB-361 | 6 |
| | MDA-MB-453 | 3.9 |
| | SKBr3 | 2.3 |
| | T47D | 2.6 |
| | MCF7 | 2.3 |
| | MDA-MB-231 | 7.7 |
| | MDA-MB-468 | 3.5 |
| Melanoma | SK-MEL-5 | 3 |
| | A375 | 3 |
| | MALME-3M | 7.7 |
| | SK-MEL-28 | 8 |
| | WM266.4 | 6.2 |
| Multiple myeloma (MM) | RPMI8226 | 36.7 |
| | U266 | 23.3 |
| Colon | BE | 2.8 |
| | Colo205 | 6.2 |
| | HCT116 | 16 |
| | HT29 | 30 |
| | MAWI | 50 |
| | RKO | 3.1 |
| Glioblastoma | U87MG | 6 |
| Head & Neck | HN5 | 8 |
| Leukemia | RPMI-8226 | 6.3 |
| Lung | A549 | 11.7 |
| | MV522 | 8.1 |
| | NCI-H1299 | 5.7 |
| | NCI-H460 | 14 |
| Ovarian | 41M | 3 |
| | A2780 | 6.1 |
| | CH1 | 2.8 |
| | SKOV3 | 3.7 |
| Prostate | PC3 | 5 |
| Stomach | NCI-N87 | 0.2 |
| Gastrointestinal | MO7e | 10.6 |
| stromal tumour (GIST) | GIST882 | 6.2 |
| | Baf3 | 22.4 |

### EXAMPLE 2

### In Vitro Effects of 5-(2,4-Dihydroxy-5-isopropyl-phenyl)-4-(4-morpholin-4-ylmethyl-phenyl)-isoxazole-3-Carboxylic Acid Ethylamide (AUY922) on a Panel of Primary Human Tumor Cells

The anticancer activity of 5-(2,4-Dihydroxy-5-isopropyl-phenyl)-4-(4-morpholin-4-ylmethyl-phenyl)-isoxazole-3-carboxylic acid ethylamide is evaluated in 30 human tumor xenografts in vitro using a clonogenic assay. In this assay, human cells derived from cancer patients are evaluated for the capacity of 5-(2,4-Dihydroxy-5-isopropyl-phenyl)-4-(4-morpholin-4-ylmethyl-phenyl)-isoxazole-3-carboxylic acid ethylamide to inhibit the formation of 3 dimensional colonies. These consist of tumor cells that possess the potential for anchorage independent growth in semisolid medium. The tumor xenografts which have never been cultured in cell culture plastic dishes are isolated from nude mice. Tumor cell suspensions are prepared and incubated in 24 well plates containing layers of soft agar. Under these conditions a special subpopulation of cells selectively grows to colonies. 5-(2,4-Dihydroxy-5-isopropyl-phenyl)-4-(4-morpholin-4-ylmethyl-phenyl)-isoxazole-3-carboxylic acid ethylamide was tested in 6 concentrations up to 10 pM. The tumor test panel comprises 1 to 6 models of 10 different human tumor or cancer types, which were bladder cancer, colon, liver, non small cell lung (adeno, squamous epithelium and large cell), small cell lung, mammary, ovary, pancreatic, melanoma and pleuramesothelioma. Antitumor effects are recorded as inhibition of colony formation in relation to untreated controls. The concentration which results in 50% reduction in colony formation (IC₅₀) are shown in Table 2. Further information on the method has been published (Burger et al., 2004; Fiebig et al., 2004; Smith et al., 2005).

Table 2 shows the concentration (nM) of 5-(2,4-Dihydroxy-5-isopropyl-phenyl)-4-(4-morpholin-4-ylmethyl-phenyl)-isoxazole-3-carboxylic acid ethylamide which inhibits colony formation by 50% (IC₅₀). The cells are continually exposed to 5-(2,4-Dihydroxy-5-isopropyl-phenyl)-4-(4-morpholin-4-ylmethyl-phenyl)-isoxazole-3-carboxylic acid ethylamide and colony formation is determined.

TABLE 2

| | Tumor | | IC50 |
|---|---|---|---|
| Tumor Type | model | Histology | (nM) |
| Bladder | BXF 1218 | Transitional cell carcinoma | 27 |
| | BXF 1228 | Transitional cell carcinoma | 630 |
| Colon | CXF 1103 | Adeno carcinoma | 13 |
| | CXF 158 | Adeno carcinoma | 369 |
| | CXF 1729 | Carcinoma | 467 |
| | CXF 1784 | Carcinoma | 418 |
| | CXF 609 | Adeno carcinoma | 55 |
| Liver | LIXF 575 | Hepatocellular carcinoma | 34 |
| Lung, non- | LXFA 297 | Adeno carcinoma | 28 |
| small cell | LXFA 526 | Adeno carcinoma | 5 |
| | LXFA 629 | Adeno carcinoma | 35 |
| | LXFA 983 | Adeno carcinoma | 126 |
| | LXFE 1422 | Squamous cell carcinoma | 48 |
| | LXFL 1647 | Large cell lung carcinoma | 34 |
| Lung, | LXFS 615 | Small cell lung carcinoma | 30 |
| small cell | LXFS 650 | Small cell lung carcinoma | 2 |
| Breast | MAXF 1162 | Invasive ductal carcinoma | 304 |
| | MAXF 1322 | Pap. adeno carcinoma | 29 |
| | MAXF 1384 | Adeno carcinoma | 209 |
| | MAXF 401 | Pap. adeno carcinoma | 78 |
| | MAXF 583 | Ductual adeno carcinoma | 333 |
| Melanoma | MEXF 1539 | Melanoma | 3 |
| | MEXF 462 | Amelanotic melanoma | 24 |
| | MEXF 535 | Amelanotic melanoma | 43 |
| | MEXF 672 | Amelanotic melanoma | 18 |
| | MEXF 989 | Amelanotic melanoma | 2 |
| Ovary | OVXF 1353 | Adeno carcinoma | 26 |
| | OVXF 1544 | Carcinoma | 53 |
| Pancreas | PAXF 1657 | Adeno carcinoma | 39 |
| Pleuramesothelioma | PXF 1118 | Biphasic | 223 |
| | | pleuramesothelioma | |

### EXAMPLE 3

### Antitumor Effect of 5-(2,4-Dihydroxy-5-isopropyl-phenyl)-4-(4-morpholin-4-ylmethyl-phenyl)-isoxazole-3-Carboxylic Acid Ethylamide (AUY922) in the Human Breast Cancer Model BT-474

The estrogen receptor positive cell line BT-474 was initially isolated from a human breast ductal carcinoma established from a solid, invasive ductal carcinoma of the breast obtained from a 60-year-old woman (ATCC number HTB-20). The cells are grown in DMEM high glucose (4.5 g/l) supplemented with 10% FCS, 200 mM L-glutamine and 1% sodium pyruvate.

In preparation for cell inoculation, each mouse is subcutaneously implanted on the upper dorsal side with a 17β-Estradiol pellet (25 μg/day; 90 day release) using a trocar needle. BT-474 cells (5×10̂6) are injected in 200 μl Matrigel:HBSS (1:1 vol) (BD MatrigelTM Basement Membrane Matrix). The injection site is subcutaneously in the right flank. Treatment with AUY922 is initiated when the average tumor volume reached approximately 100 mm³. Tumor growth is monitored at regular intervals. The xenograft tumor sizes are measured manually with calipers and the tumor volume is estimated using the formula: (W×L×H×π/6), where width (W), height (H) and length (L) are the three largest diameters. Results are presented as mean ±SEM. Tumor data are analyzed by ANOVA with post hoc Dunnet's test for comparison of treatment versus control group. As a measure of efficacy the % T/C value is calculated at the end of the experiment according to:

(Δtumor volume_{treated}/Δtumor volume_{control})*100

where Δtumor volumes represent the mean tumor volume on the evaluation day minus the mean tumor volume at the start of the experiment.

The antitumor effect of AUY922 is evaluated in the BT-474 xenograft model. In this study, the treatment period is 21 days. Each group consists of eight tumor bearing animals. At the end of the study, the tumor sizes in the treatment groups are compared to those of the vehicle treated groups and the effect is expressed as % T/C. Statistically significant reduction of tumor sizes are observed when AUY922 is administered once per week at 17-25 mg/kg (Table 3).

TABLE 3
Effect of AUY922 on BT-474 xenograft growth

| | | | ΔTumor volume |
|---|---|---|---|
| Compound | Dose, schedule, route | T/C (%) | (mm³) |
| Vehicle | 10 ml/kg, qw, i.v. | 100 | 528 ± 123 |
| control | | | |
| AUY922 | 8.3 mg/kg, qw, i.v. | 43 | 229 ± 73 |
| AUY922 | 17 mg/kg, qw, i.v. | 9 | 46 ± 27* |
| AUY922 | 25 mg/kg, qw, i.v. | 3 | 15 ± 23* |

*P < 0.05; one-way ANOVA post hoc Dunnet's test.

### EXAMPLE 4

### Antitumor Effect of 5-(2,4-Dihydroxy-5-isopropyl-phenyl)-4-(4-morpholin-4-ylmethyl-phenyl)-isoxazole-3-Carboxylic Acid Ethylamide (AUY922) in the Rat Breast Cancer Model BN-472

The transplantable rat breast cancer tumor BN472 is serially passaged as fragments in female syngeneic Brown Norway rats. The injection site is orthotopically in the mammary fat pad. Treatment with AUY922 is initiated when the average tumor volume reaches approximately 100 mm³. Tumor growth is monitored at regular intervals. The xenograft tumor sizes are measured manually with calipers and the tumor volume is estimated using the formula: (W×L²×π/6), where width (W) and height (H) are the two largest diameters. Results are presented as mean ±SEM. Tumor data were analyzed by ANOVA with post hoc Dunnet's test for comparison of treatment versus control group. As a measure of efficacy the % T/C value is calculated at the end of the experiment according to:

(Δtumor volume_{treated}/Δtumor volume_{control})*100

where Δtumor volumes represent the mean tumor volume on the evaluation day minus the mean tumor volume at the start of the experiment.

The antitumor effect of AUY922 is evaluated in the BN472 xenograft model. Each group consists of seven tumor bearing animals. At the end of the study, the tumor sizes in the treatment groups are compared to those of the vehicle treated groups and the effect is expressed as %T/C. Statistically significant reduction of tumor sizes is observed when AUY922 was administered once per week at 50 mg/kg (Table 4).

TABLE 4
Effect of AUY922 on BN472 xenograft growth

| | | | ΔTumor volume |
|---|---|---|---|
| Compound | Dose, schedule, route | T/C (%) | (mm³) |
| Vehicle | 2 ml/kg, qw, i.v. | 100 | 5569 ± 1639 |
| control | | | |
| AUY922 | 25 mg/kg, qw, i.v. | 78 | 4357 ± 1338 |
| AUY922 | 50 mg/kg, qw, i.v. | 21 | 1148 ± 152* |

*P < 0.05; one-way ANOVA post hoc Dunnet's test.

### EXAMPLE 5

### Antitumor Effect of 5-(2,4-Dihydroxy-5-isopropyl-phenyl)-4-(4-morpholin-4-ylmethyl-phenyl)-isoxazole-3-Carboxylic Acid Eethylamide (AUY922) in the Rat Pancreatic Cancer Model CA20948

The transplantable rat pancreatic tumor CA20948 is serially passaged as cell homogenates in male syngeneic Lewis rats. The injection site is subcutaneously on the right flank.

Treatment with AUY922 is initiated when the average tumor volume reaches approximately 100 mm³. Tumor growth is monitored at regular intervals. The xenograft tumor sizes is measured manually with calipers and the tumor volume is estimated using the formula: (W×L²×π/6), where width (W) and height (H) are the two largest diameters.

Results are presented as mean ±SEM. Tumor data were analyzed by ANOVA with post hoc Dunnet's test for comparison of treatment versus control group. As a measure of efficacy the % T/C value is calculated at the end of the experiment according to:

(Δtumor volume_{treated}/Δtumor volume_{control})*100

where Δtumor volumes represent the mean tumor volume on the evaluation day minus the mean tumor volume at the start of the experiment.

The antitumor effect of AUY922 is evaluated in the CA20948 xenograft model. Each group consisted of six tumor bearing animals. At the end of the study, the tumor sizes in the treatment groups are compared to those of the vehicle treated groups and the effect is expressed as % T/C. Statistically significant reduction of tumor sizes is observed when AUY922 is administered once per week at 50 and 75 mg/kg (Table 5).

TABLE 5
Effect of AUY922 on CA20948 xenograft growth

| | | | ΔTumor volume |
|---|---|---|---|
| Compound | Dose, schedule, route | T/C (%) | (mm³) |
| Vehicle | 2 ml/kg, qw, i.v. | 100 | 23267 ± 7810 |
| control | | | |
| AUY922 | 50 mg/kg, qw, i.v. | 30 | 7090 ± 2553* |
| AUY922 | 75 mg/kg, qw, i.v | 21 | 4796 ± 1354* |

*P < 0.05; one-way ANOVA post hoc Dunnet's test.

1. A method of treating humans suffering from cancer of the bladder, the colon, the liver, the lung, the breast, the ovaries, the pancreas, the kidney, the stomach, the gastrointestinal tract, the prostate, the head and neck, the brain, and/or the blood which comprises administering to said human in need of such treatment a dose of 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate, effective against cancer of the bladder, the colon, the liver, the lung,the breast, the vagina, the ovaries, the pancreas, the kidney, the stomach, the gastrointestinal tract, the prostate, the head and neck, the peritoneal cavity, the thyroid, the bone, the brain, the central nervous system and/or the blood and/or myelodysplastic syndrome, systemic mastocytosis, von Hippel-Lindau syndrome, multicentric Castleman disease and/or psioriasis.

2. A pharmaceutical preparation for the treatment of cancer of the bladder, the colon, the liver, the lung,the breast, the vagina, the ovaries, the pancreas, the kidney, the stomach, the gastrointestinal tract, the prostate, the head and neck, the peritoneal cavity, the thyroid, the bone, the brain, the central nervous system and/or the blood and/or myelodysplastic syndrome, systemic mastocytosis, von Hippel-Lindau syndrome, multicentric Castleman disease and/or psioriasis comprising 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate and at least one pharmaceutically acceptable carrier.

3. Method according to claim 3 wherein a weekly dose of 2 to 300 mg of 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate is administered to a human.

4. A method for administering to a human subject having cancer of the bladder, the colon, the liver, the lung,the breast, the vagina, the ovaries, the pancreas, the kidney, the stomach, the gastrointestinal tract, the prostate, the head and neck, the peritoneal cavity, the thyroid, the bone, the brain, the central nervous system and/or the blood and/or myelodysplastic syndrome, systemic mastocytosis, von Hippel-Lindau syndrome, multicentric Castleman disease and/or psioriasis 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate, which comprises administering a pharmaceutically effective amount of 5-(2,4-Dihydroxy-5-isopropyl-phenyl)-4-(4-morpholin-4-ylmethyl-phenyl)-isoxazole-3-carboxylic acid ethylamide or a tautomer thereof or a pharmaceutically acceptable salt or a hydrate or a solvate to the human subject once weekly or more frequently.
